(19) United States
(12) Patent Application Publication
(10) Pub. No.: US 2015/0202076 A1
(43) Pub. Date: Jul. 23, 2015

(54) Intrauterine Device

(71) Applicant: Mithra Pharmaceuticals S.A., Liege (BE)

(72) Inventor: Fabienne Wijzen, Lince (BE)

(21) Appl. No.: 14/420,275

(22) PCT Filed: Aug. 8, 2013
(86) PCT No.: PCT/EP2013/066619

§ 371 Date: Feb. 6, 2015

Related U.S. Application Data
(60) Provisional application No. 61/681,282, filed on Aug. 9, 2012.

(30) Foreign Application Priority Data
Aug. 9, 2012 (EP) 12179886.2

(51) Int. Cl. A61F 6/18; A61F 6/14

(52) U.S. Cl. CPC A61F 6/18; A61F 6/144

(57) ABSTRACT

The present invention concerns an intrauterine device (100), IUD, comprising a stem element (30), a cross-member (70), and a withdrawal string (90), wherein the stem element (30) comprises a hollow (34) elongated body (32); the cross-member (70) comprises a coupling element (74) configured for dismountable attachment to the stem element (30), and one or more arms (76) attached to the coupling element (74); and the withdrawal string (90) is anchored to the cross-member (70) and is threaded through the hollow (34) of the stem element (30).

### FIELD OF THE INVENTION

The present invention relates to an intrauterine device (IUD). In particular, the present invention relates to a composition-eluting IUD.

### BACKGROUND OF THE INVENTION

Copper or hormonal intra-uterine devices (IUDs) or intra-uterine systems (IUSs) are used as a common method of contraception and/or for treatment of menorrhagia and/or for treatment of other conditions. The term IUD (intra-uterine device) will be used herein to refer to both IUDs and IUSs.

IUDs for the delivery of hormones and methods for their preparation have been described.

For instance, WO 03/068117 describes a T-shaped intra-uterine device. At least the stem of the T is made of a fiber releasing an active substance while the arms are attached to the stem. The stem can be made of several fiber segments, each of which releases a different active substance, and the rod forming the arms can also be made of a fiber releasing an active substance.

For instance, EP 1 400 258 describes a delivery system comprising a body construction and at least one capsule containing a pharmaceutical composition, said capsule having at least a first end and a second end. The body construction has at least two locking parts, each locking part having a surface adapted to face and cover one of the at least first and second ends of the capsule, and in that the capsule is mounted between said at least two locking parts. EP 1 400 258 further describes one manufacturing process of a delivery system, said system comprising a body construction and at least one capsule containing a pharmaceutical composition.

For instance, WO 91/07934 describes a process for the manufacture of an intra-uterine device provided with a withdrawal string. The IUD is an essentially T-shaped plastic element with a middle leg. The thread is laid in the injection mould in the lengthwise direction of the middle leg of the IUD. Then the mould is closed and the IUD is subsequently produced by injection moulding, following which the IUD is removed from the mould. A coil or a product which is to be absorbed slowly by the body is placed on the middle leg.

For instance, EP 0 673 629 describes an intrauterine contraceptive device comprising at least two flexible arms, extending from a central point characterized in that the tips of the flexible arms, which are solid, are directed toward the fungus, whereby the arms from the central point to about the massive tips along the front and back are provided with cavities, while the central point is provided with a flexible thread, surrounded by the means acting as contraceptive, while the thread is provided with a holding device for the means acting as contraceptive.

WO 2007/075086 discloses a T-shaped intrauterine contraceptive device having two flexible arms, which is provided with a flexible wire stem surrounded with an active substance. The IUD requires a wide diameter stem to maintain contact with the arms, and retain integrity of the T-shape.

The major disadvantages of the aforementioned hormonal IUDs include problems to withdraw the IUD from the intrauterine cavity due to limited mechanical resistance to traction and the size of the IUDs which may complicate their insertion into and/or withdrawal from the intrauterine cavity. Withdrawal of the IUD from the intrauterine cavity is required when the IUD is no longer active or its presence is no longer desired. Removal of prior art IUDs from the intrauterine cavity may be problematic due to the incapacity of the structure to withstand the forces required to retract the IUD from the intrauterine cavity. Additionally, the size of the IUD affects the ability for all women to benefit; larger IUDs require inserters having a large diameter which will exclude use by several female population groups.

In view of the above, it is an object of the present invention to provide an IUD with reduced dimensions together with a good resistance to traction compared with prior art IUDs. Furthermore, it is an object of the present invention to provide further and/or improved IUDs which allow easy insertion into and removal from the intrauterine cavity by the medical practitioner.

### SUMMARY OF THE INVENTION

The present inventors have now found an IUD addressing one or more of the above-mentioned problems of the prior art.

Hence, the present invention relates to an intrauterine device, IUD, preferably a composition-eluting IUD, comprising a stem element, a cross-member, and a withdrawal string, wherein:
the stem element comprises a hollow elongated body;
the cross-member comprises a coupling element for dismountable attachment to the stem element, and one or more arms attached to the coupling element; and
the withdrawal string is anchored to the cross-member and is threaded through the hollow of the stem element.

In particular, the coupling element is configured for dismountable attachment to the stem.

The withdrawal string (90) may be anchored to the coupling element (74). The stem element (30) and the cross-member (70) may form a continuous structure. The withdrawal string (90) may maintain the stem element (30) in an abutting alignment with the cross-member (70). The withdrawal string (90) may comprise a stop member (92) configured to maintain the stem element (30) and the cross-member (70) in abutting alignment. The stop member (92) may be a knot. The stop member (92) may maintain the stem element (30) and the cross-member (70) in a continuous structure. The IUD may be a composition-eluting IUD. The IUD may be one that elutes a composition. The stem element (30) may at least partially be formed from a polymer comprising the composition. The polymer may be a slow-release polymer. The polymer may be adapted to prolong and/or control the release of the composition from the IUD. The polymer may be formed as a polymeric core (36) comprising the composition, disposed with an outer membrane (38) configured to control release of the composition. The composition may comprise at least one active pharmaceutical ingredient. The elongated body (32) may have an outer diameter equal to or less than 2.2 mm. The elongated body (32) may be cylindrical and have an outer diameter equal to or less than 2.2 mm. The IUD may have a tensile strength of at least 9N. The coupling element (74) may comprise a passage (78) configured to fittingly receive a distal end (31) of the hollow elongated body (32) of the stem element (30). The coupling element (74) may comprise a passage (78) configured to slideably receive a distal end (31) of the hollow elongated body (32) of the stem element (30). The withdrawal string (90) may comprise a stop member (92) configured to maintain the stem element (30) coupled to the coupling element (74) of the cross-member (70). The stem element (30) may be essentially rigid.

Such an IUD advantageously facilitates removal of the IUD from the intrauterine cavity, even after long-term implantation. Due to the design of the present IUD, the forces needed for removal of the IUD from the intrauterine cavity become compression forces on the stem element, thereby reducing the risk of product failure such as breakage and allowing easy withdrawal of the IUD. Also, the design of the present IUD largely protects the withdrawal string from the physical environment, i.e., the intrauterine cavity, thereby prolonging its mechanical integrity and facilitating withdrawal of the IUD from the intrauterine cavity, even after long-term implantation.

Furthermore, by anchoring the withdrawal string to the cross-member, much lower tensile strength of the stem element is required compared with when the withdrawal string is attached to the proximal end of the stem element. Therefore, an IUD embodying the principles of the present invention advantageously may have reduced dimensions compared to prior art IUDs while being suitable to contain the same amount or even more drugs for long-term implantation and use. The reduced diameter of the stem element of the IUD allows a reduced diameter of the inserter tube and hence, facilitates insertion of the IUD in the uterus. Additionally, the reduced diameter of the IUD increases the comfort of the user.

These and further aspects and embodiments of the invention are hereunder further explained in the following sections and in the claims, and illustrated by non-limiting figures. The reference numbers relate to the hereto-annexed figures.

### BRIEF DESCRIPTION OF THE FIGURES

FIG. 1 schematically illustrates an intrauterine device (IUD) according to an embodiment of the invention.

FIG. 2 schematically illustrates a cross-member according to an embodiment of the invention.

FIG. 3 schematically illustrates a stem element according to an embodiment of the invention.

FIG. 4A, FIG. 4B, and FIG. 4C schematically illustrate an enlarged longitudinal (medial) cross-section of a part of a cross-member according to certain embodiments of the invention.

FIG. 5 schematically illustrates an enlarged transversal cross-section of a part of a stem element according to an embodiment of the invention.

FIG. 6A and FIG. 6B schematically illustrate an enlarged transversal cross-section of a part of an IUD according to certain embodiments of the invention.

FIG. 7 schematically illustrates a transversal cross-section of an IUD according to an embodiment of the invention.

### DETAILED DESCRIPTION OF THE INVENTION

Unless defined otherwise, all technical and scientific terms used herein have the same meaning as is commonly understood by one of skill in the art. All publications referenced herein are incorporated by reference thereto.

The articles ‘a’ and ‘an’ are used herein to refer to one or to more than one, i.e. to at least one of the grammatical object of the article.

Throughout this application, the term ‘about’ is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of elements). The recitation of end points also includes the end point values themselves (e.g. from 1.0 to 5.0 includes both 1.0 and 5.0).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The terms “distal”, “distal end”, “proximal” and “proximal end” are used through the specification, and are terms generally understood in the field to mean towards (proximal) or away (distal) from the practitioner side of the device. Thus, “proximal (end)” means towards the practitioner side and, therefore, away from the subject or patient side. Conversely, “distal (end)” means towards the subject or patient side and, therefore, away from the practitioner side.

In the following detailed description of the invention, reference is made to the accompanying drawings that form a part hereof, and in which are shown by way of illustration only of specific embodiments in which the invention may be practiced. It is to be understood that other embodiments may be utilized and structural or logical changes may be made without departing from the scope of the present invention.

The present invention provides an intrauterine device, IUD, comprising a stem element, a cross-member, and a withdrawal string, wherein: the stem element comprises a hollow elongated body; the cross-member comprises a coupling element for dismountable attachment to the stem element, and one or more arms attached to the coupling element; and the withdrawal string is anchored to the cross-member and is threaded through the hollow of the stem element. The IUD is preferably one that elutes a composition. In particular, the stem element is one that elutes a composition.

In an embodiment, the stem element comprises a hollow elongated body and the withdrawal string is threaded through the hollow of the stem element.

The term “stem element”, as used herein, refers to an element that forms the body of the IUD, typically of a generally T-shaped or generally Y-shaped IUD.

The elongated body is preferably movably or slideably mounted on the withdrawal string. The elongated body is preferably movably or slideably mounted on the withdrawal string, whereby the sliding movement is limited or abolished by a stop member on the withdrawal string.

Advantageously, the design of the present IUD allows the stem element to have a reduced size while still being able to contain sufficient composition for the period of implantation. The stem element may be essentially rigid.

In an embodiment, the stem element and the cross-member form a continuous structure. Preferably, it is not articulated or discontinuous. In a preferred embodiment, the stem element and the cross-member form a continuous fixed structure, i.e., the structure is made of non-jointed elements. The stop member of the withdrawal string preferably keeps the stem element and the cross-member in a continuous structure.

The recitation “continuous structure” is meant to encompass that the cross-member and the stem element form an integral or uninterrupted structure, i.e., the structure is not articulated or discontinuous.

Advantageously, such a continuous structure provides that no or little frictional or tensile forces are present between the elongated body and the withdrawal string. Furthermore, such a continuous structure provides that at least a part of the withdrawal string is protected by the cross-member and elongated body from the surrounding environment.

The stem element may have any outer transverse profile such as circular, oval, polygonal, or other geometric shape. Preferably, the stem element is cylindrical, having a circular outer transverse profile.

The elongated body may have an outer diameter of about 1.5 mm, 1.6 mm, 1.7 mm, 1.8 mm, 1.9 mm, 2.0 mm, 2.1 mm, 2.2 mm, 2.3 mm, 2.4 mm, or 2.5 mm, or a value between any two of the aforementioned values. The elongated body may have an outer diameter ranging of from about 1.5 mm to about 2.5 mm, for example, the elongated body may have an outer diameter ranging of from about 1.8 mm to about 2.2 mm. Preferably, the elongated body has an outer diameter equal to or less than 2.2 mm.

The elongated body may have a length ranging of from about 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, or a value between any two of the aforementioned values. The elongated body may have a length ranging of from about 16 mm to about 32 mm, for example, the elongated body may have a length ranging of from about 18 mm to about 30 mm, for example, the elongated body may have a length ranging of from about 20 mm to about 28 mm. Preferably, the elongated body may have a length equal to or less than 26 mm

Such reduced dimensions of the stem element advantageously facilitate insertion of the IUD into and/or removal from the intrauterine cavity by the medical practitioner while still allowing to contain sufficient composition such as active pharmaceutical ingredient for the time of implantation.

The hollow of the elongated body is preferably provided as a lumen that spans the length of the elongated body.

The hollow is preferably open at both ends of the elongated body, i.e., the distal end of the elongated body and the proximal end of the elongated body.

The inner diameter of the hollow is preferably larger than the outer diameter of the withdrawal string. The withdrawal string is preferably configured to advance through the hollow.

The hollow of the elongated body may have an inner diameter of about 0.25 mm, 0.3 mm, 0.4 mm, 0.5 mm, 0.6 mm, 0.7 mm, 0.8 mm, 0.9 mm, or 1 mm, or a value in the range between any two of the aforementioned values. The hollow of the elongated body may have an inner diameter ranging of from about 0.25 mm to about 1 mm, for example, the hollow may have a diameter ranging of from about 0.4 mm to about 0.8 mm.

In an embodiment, the stem element comprises a hollow elongated body comprising a polymer or polymers for release of the composition. The stem element may be at least partially or entirely formed from a polymer. The polymer may be any biocompatible polymer, either biodegradable or non-biodegradable. The polymer may be a slow release polymer. Advantageously, such a slow-release polymer may allow to prolong and/or control the release of the composition from the IUD. The stem element may be at least partially or entirely formed from a polymeric core and an outer membrane; in other words the polymer may be a polymeric core disposed with an outer membrane.

In an embodiment of the present IUD, the stem element comprises or is at least partially formed from a polymeric core and an outer membrane. Preferably, the stem element comprises or is at least partially formed from a polymeric core or reservoir, for containing the composition, and an outer membrane or sleeve, for controlling diffusion of the composition. An example of a suitable construction is a combination of a polymeric core and an outer membrane, wherein the polymeric core comprises at least one composition and is encased in an outer membrane. The stem element may comprise one or more outer membranes to improve controlling diffusion of the composition.

In an embodiment, the stem element may be at least partially formed from a polymer comprising a composition. The polymer preferably is formed as a polymeric core comprising the composition, disposed with an outer membrane configured to control release of the composition. The delivery rate of the composition may be controlled either by the polymeric core or by the outer membrane, or by both of them.

In an embodiment, the polymeric core comprises a polymer such as a slow-release polymer. In an embodiment, the outer membrane comprises a polymer such as a slow-release polymer. Suitable non-limiting polymers include polyethylene, polypropylene, polymethylpentene ethylene/propylene copolymers, ethylene/ethyl acrylate copolymers, ethylene/vinyl acetate (EVA) copolymers, polycarbonate, polytetrafluoroethylene (PTFE), fluoroethylenepropylene (FEP), polyvinylidene fluoride (PVDF), polyvinylacetate, polystyrene, polyamides, polyurethane, polybutadiene, polyisoprene, chlorinated polyethylene, polyvinyl chloride, vinyl chloride copolymers with vinyl acetate, poly(methacrylate), polymethyl (meth)acrylate, poly(vinylidene) chloride, poly(vinylidene) ethylene, poly(vinylidene) propylene, polyethylene terephthalate, ethylene vinylacetate, a polyhydroxy alkoanate poly(lactic acid), poly(glycolic acid), poly(alkyl 2-cyanoacrylates), polyanhydrides, polyorthoesters, ethylene/vinyl alcohol copolymer, ethylene/vinyl acetate/vinyl alcohol terpolymer; ethylene/vinyloxyethanol copolymer, hydrophilic polymers such as the hydrophilic hydrogels of esters of acrylic and methacrylic acids, modified collagen, cross-linked polyvinyl alcohol, cross-linked, partially hydrolyzed polyvinyl acetate, silicone elastomers, especially the medical grade polydimethyl siloxanes, polyvinylmethylsiloxanes, other organopolysiloxanes, polysiloxane, neoprene rubber, butyl rubber, epichlorohydrin rubbers, hydroxyl-terminated organopolysiloxanes of the room temperature vulcanizing type which harden to elastomers at room temperature following the addition of cross-linking agents in the presence of curing catalysts, two-component dimethylpolysiloxane compositions which are platinum catalysed at room temperature or under elevated temperatures and capable of addition cross-linking as well as mixtures thereof.

Especially suitable polymers are an elastomer composition comprising poly(dimethylsiloxane) (PDMS), an elastomer composition comprising a siloxane-based elastomer comprising 3,3,3-trifluoropropyl groups attached to the Si-atoms of the siloxane units, an elastomer composition comprising poly(alkylene oxide) groups, said poly(alkylene oxide) groups being present as alkoxy-terminated grafts or blocks linked to the polysiloxane units by silicon-carbon bonds, or as a mixture of these forms and a combination of at least two thereof.

Preferably, the polymer is selected from the group consisting of an ethylene/vinyl acetate (EVA) copolymer, polyurethane (PU), and silicone.

Preferably, the polymeric core comprises a polymer selected from the group consisting of an ethylene/vinyl acetate (EVA) copolymer, polyurethane (PU), and silicone.

Preferably, the outer membrane comprises a polymer selected from the group consisting of an EVA copolymer, PU, and silicone.

The polymeric core may have a volume, i.e., outer diameter and length to comprise sufficient composition for the time of implantation such as for example during at least three years. The polymeric core may have an outer diameter ranging of about 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, or 2.5 mm or a value between any two of the aforementioned values. The polymeric core may have an outer diameter ranging of from about 1.4 mm to about 2.4 mm, or from about 1.5 mm to about 2.5 mm, for example, the polymeric core may have an outer diameter ranging from about 1.7 mm to about 2.1 mm, or from about 1.8 mm to about 2.2 mm. Preferably, the polymeric core may have an outer diameter equal to or less than 2.1 mm or 2.2 mm. Preferably, the polymeric core has an outer diameter of about 1.9 mm or about 2 mm.

The polymeric core may have a length of about 16 mm, 17 mm, 18 mm, 19 mm, 20 mm, 21 mm, 22 mm, 23 mm, 24 mm, 25 mm, 26 mm, 17 mm, 28 mm, 29 mm, 30 mm, 31 mm, 32 mm or a value in the range between any two of the aforementioned values. The polymeric core may have a length ranging of from about 16 mm to about 32 mm, for example, the polymeric core may have a length ranging of from about 18 mm to about 30 mm, for example, the polymeric core may have a length ranging of from about 20 mm to about 28 mm. Preferably, the polymeric core has a length equal to or less than 26 mm.

The outer membrane may have a thickness to control the diffusion of the composition such as the active pharmaceutical ingredient. The outer membrane may have a thickness of about 10 μm, 25 μm, 50 μm, 100 μm, 200 μm, 300 μm, 400 μm, 500 μm, 600 μm, 700 μm, 750 μm, 800 μm, 900 μm or 1000 μm or a value in the range between any two of the aforementioned values. The outer membrane may have a thickness ranging form about 10 μm to about 1000 μm, for example, the outer membrane may have a thickness ranging form about 25 μm to about 750 μm, for example, the outer membrane may have a thickness ranging form about 50 μm to about 400 μm.

The stem element is preferably made by co-extrusion. The polymeric core and the outer membrane may be produced together by co-extrusion. Or, the polymeric core may be produced by casting or molding of the elongated body and afterwards assembling the elongated body with the outer membrane produced by extrusion.

The stem element may further comprise an end piece at the proximal end of the elongated body. In an embodiment, the end piece comprises a body containing a hollow passage which spans the length of the end piece. The body may be flattened. The outer transverse profile of the body is preferably the same as that of the stem element. The end piece preferably is provided at the proximal end of the elongated body by means of the withdrawal string threaded through the hollow of the end piece. Advantageously, the end piece may maintain the stem element and the cross-member in a continuous structure.

The end piece may be substantially made of any polymeric material which is tolerated by the uterus. The end piece may be substantially made of any biocompatible polymer. Preferably, the end piece is substantially made of polyethylene.

The end piece may have substantially the same outer diameter as the elongated body. The end piece may have an outer diameter of about 1.5 mm, 1.6 mm, 1.7 mm, 1.8 mm, 1.9 mm, 2.0 mm, 2.1 mm, 2.2 mm, 2.3 mm, 2.4 mm, or 2.5 mm or a value between any two of the aforementioned values. The end piece may have an outer diameter ranging of from about 1.5 mm to about 2.5 mm, for example, the end piece may have an outer diameter ranging of from about 1.8 mm to about 2.2 mm. Preferably, the end piece has an outer diameter equal to or less than 2.2 mm.

The end piece may have a length of about 1 mm, 2 mm, 3 mm, 4 mm, 5 mm, or a value between any two of the aforementioned values. The end piece may have a length ranging of from about 1 mm to about 5 mm, for example, the end piece may have a length ranging of from about 2 mm to about 4 mm. Preferably, the end piece has a length ranging of from about 2.2 mm to about 3 mm.

In an embodiment of the present IUD, the stem element may comprise a composition. In an embodiment, the IUD may be a composition-eluting IUD.

In certain embodiments, the polymeric core of the stem element may comprise a polymer and a composition. In certain embodiments, the polymer of the stem element comprises a composition.

The composition may comprise, consist essentially of, or consist of at least one active pharmaceutical ingredient. The composition may comprise, consist essentially of, or consist of at least one non-active pharmaceutical ingredient. The composition may comprise a combination of at least one active and at least one non-active pharmaceutical ingredient.

The at least one active pharmaceutical ingredient may be selected from the group consisting of contraceptives, hormones, steroids, drugs for hormone replacement therapy, selective androgen receptor modulators (SARM), drugs for the treatment of premenstrual syndrome, drugs for the treatment of endometriosis, drugs for the treatment of uterine fibroids including uterine leiomyomata and leiomyosarcoma, drugs for cervical ripening and/or induction of labour, selective estrogen receptor modulators (SERMs), selective progestin receptor modulators (SPRM), antimalarial substances, osteoporosis drugs, antiprogestins, aromatase inhibitors, bone active substances, antiurinary incontinence substances, serotonin reuptake inhibitors (SSRIs), drugs for genitourinary disorders, anti-emetic drugs, 5HT3 antagonists, anti-angiogenesis factors, growth factors, enzymes, anesthetics, analgesics, anticoagulants and thrombolytic substances, anti-inflammatory substances, antimicrobials, anti-protozoal substances, antiviral substances, neuroleptic and antipsychotic drugs, opiate antagonists and agonists, anti-fibroid substances, antihypertensives, angiotensin inhibitors, anti-protozoal substances, anti-addiction drugs, anti-angiogenesis factors, anti-bacterial substances, anticancer chemotherapeutic substances, antifungals, antioxidants, diuretics, drugs for the central nervous system, fibrinolytic substances, free radical scavengers, gene therapy substances, growth factors, neuro-trophic factors, peptides, photodynamic therapy substances, proteins, symphatomimetic substances, thrombin inhibitors, thrombolytic substances, and a combination of at least two thereof.

The at least one active pharmaceutical ingredient may be a medicament such as a medicament for use in the treatment of menorrhagia, pain and/or other conditions such as conditions confined to the intrauterine cavity.

The composition may comprise a second active pharmaceutical ingredient, for example a substance capable of preventing or suppressing abnormal and/or irregular endometrial bleeding. Hence, the at least one active pharmaceutical ingredient may be selected from the group of prostaglandin synthesis inhibitors, NSAIDs, inhibitors of leukotriene, oxytocin antagonists, pancreatic trypsin inhibitors, COX-inhibitors, antifibrinolytic drugs, estrogens, antiestrogens, aromatase inhibitors, cytokine inhibitors, glucocorticoids, progestogens with pronounced glucocorticoid acticity, danazol, gestrinone, and angiogenesis inhibitors.

Suitable, non-limiting contraceptives include levonorgestrel (LNG), desogestrel, or progesterone.

Suitable non-limiting hormones include estrogens, progestogens, glucocorticoids and mineralocorticoids; analogs, agonists and antagonists thereof. For example, suitable estrogens include tamoxifen, oestrogen, oestradiol, ethinyl oestradiol, estron, quinestranol, estriol, estetrol and mestranol. For example, suitable progestogens include progesterone, dienogest, medroxyprogesterone acetate, norgestrel, levonorgestrel, norethindrone, norethindrone acetate, desogestrel, norgestimate, and ethynodiol diacetate, norelgestromin, 30 norethisteron, dydrogesteron, drospirenon, 3-beta-hydroxydesogestrel, etonogestrel, 17-deacetylnorgesti mat, 19-norprogesteron, acetoxypregnenolon, allylestrenol, amgeston, chlormadinon, cyproteron, demegeston, dihydrogesteron, dimethisteron, ethisteron, ethynodioldiacetat, flurogestonacetat, gastrinon, gestoden, gestrinon, hydroxymethylprogesteron, lynestrenol, mecirogeston, megestrol, melengestrol, nomegestrol, norethynodrel, norgestrienon, normethisteron, quingestanol, (17alpha)-17-hydroxy-II-methylen-19-norpregna-4,15-dien-20-yn-3-on, tibolon, trimegeston, algeston 5 acetophenid, nestoron, promegeston, 17-hydroxyprogesteronester, 19-nor-17hydroxyprogesteron, 17alpha-ethinyl-testosteron, 17alpha-ethinyl-19-nor-testosteron, d-17beta-acetoxy-13beta-ethyl-17alpha-ethinyl-gon-4-en-3-onoxim, tanaproget.

Preferably, said progestogen is progesterone or levonorgestrel (LNG).

Preferably, the composition comprises at least one active pharmaceutical ingredient selected form levonorgestrel (LNG), desogestrel, or progesterone. More preferably, the composition comprises the active pharmaceutical ingredient levonorgestrel.

In certain embodiments, the stem element may comprise a polymer selected from the group consisting of ethylene/vinyl acetate (EVA) copolymer, polyurethane (PU), and silicone and may comprise a composition comprising at least one active pharmaceutical ingredient selected form levonorgestrel (LNG), desogestrel, or progesterone, preferably LNG.

In an embodiment, the stem element comprises from about 20% to about 75% by weight of at least one active pharmaceutical ingredient as defined above. For example, the stem element comprises from about 30% to about 70% by weight of at least one active pharmaceutical ingredient, for example from about 35% to about 65% by weight, for example from about 40% to about 60% by weight of at least one active pharmaceutical ingredient, for example from about 50% to about 60% by weight of at least one active pharmaceutical ingredient.

In an embodiment, the stem element comprises the at least one pharmaceutical ingredient in such an amount so as to release the at least one active pharmaceutical ingredient in a concentration within the therapeutic window during the term of implantation such as during at least three years of implantation.

For instance, the stem element comprises levonorgestrel in such an amount so as to release levonorgestrel in a concentration ranging of from about 10 to about 25 μg per day during the term of implantation such as during at least three years of implantation.

In an embodiment, the IUD as defined herein is configured for implantation during at least one year, preferably, the IUD as defined herein is configured for implantation during at least two years, more preferably, the IUD as defined herein is configured for implantation during at least three years. For example, the IUD as defined herein is configured for implantation during three, four, five, or more years.

In an embodiment of the present IUD, the cross-member comprises one or more arms attached to a coupling element configured for dismountable attachment to the stem element.

The term “cross-member”, as used herein, refers to a traversal element that forms the arms of the IUD, typically of a generally T-shaped or Y-shaped IUD.

In an embodiment, the cross-member comprises one or more arms such as two, three, four, five, six, or more arms. Preferably, the cross-member comprises two arms.

In an embodiment, the arms may be elongated members. In a further embodiment, the arms may be bent. The extremity of each arm may be bent through an angle greater than 90° so that the extremities point to the stem element. The extremity of each arm preferably is bent through an angle of 145°. The arms may comprise a first part, a second part or extension, and a bent piece between them. The arms may have rounded tips.

The arms preferably are directed sideways from the stem element. The arms may be horizontal or may be directed slightly upwards. After deployment in the intrauterine cavity, the desired shape of the IUD may be a generally “T-shape” or “Y-shape”.

The arms are preferably symmetrically arranged around the longitudinal axis of the coupling element. The arms are preferably symmetrically arranged around the longitudinal axis of the stem element.

The arms are preferably non-composition eluting.

The arms may be flexible such as compliant or sprung such that the tips of the arms can touch when folded back. Advantageously, flexible arms allow a reduction in the size of the IUD for insertion into and/or removal from the uterus. Furthermore, flexible arms allow the IUD to return to the desired shape after deployment in the uterus.

In an embodiment, the arms are attached to the coupling element. In a preferred embodiment, the coupling element is disposed between two arms giving rise to a T-shaped IUD when the stem element is attached to the cross-member.

In an embodiment, the coupling element may be configured to retain the stem element in position in the cross-member. For example, the coupling element may retain the distal end of the elongated body in the passage of the coupling element of the cross-member. Preferably, the coupling element retains the stem element in fixed angular (directional) relation to the coupling element. Preferably, the coupling element retains the stem element in an essentially perpendicular alignment with the arms. The coupling element may allow rotation of the stem element along its longitudinal axis. The coupling element may allow translation of the stem element along its longitudinal axis. Translation of the stem element along its longitudinal axis may be limited, reduced or abolished for example by the withdrawal string comprising a stop member such as a knot.

It is advantageous to provide an effective and robust IUD that is insertable using an inserter having a minimized diameter. Certain population groups are prevented from receiving an IUD because the size of the IUD requires a larger diameter inserter. The coupling element maintains the verticality of the stem, and is effective for narrow stems. Maintaining an essentially T-configuration retains the IUD in the uterus for long-term implantation. Additionally, the coupling permits some flexing of the tip of the stem, without undue stress being placed on the coupling end of stem.

In an embodiment, the coupling element may comprise a passage or opening configured to receive the elongated body, in particular, the distal end of the elongated body. In a preferred embodiment, the coupling element may comprise a passage or opening configured to fittingly receive the elongated body, in particular, the distal end of the elongated body. The coupling element may comprise a passage or opening configured to receive or fittingly receive at least part of the distal end of the elongated body. Preferably, the coupling element frictionally retains the elongated body. The passage may partially or fully pass through a body of the coupling. The coupling element is preferably configured to retain the elongated body and cross member in an essentially planar configuration. The coupling element is preferably configured to retain the elongated body essential vertical with respect to a horizontal cross member. These configurations refer to the IUD in an open state.

In an embodiment, the coupling element may comprise a passage or opening configured to slideably receive the elongated body, in particular, the distal end of the elongated body. In an embodiment, the coupling element may comprise a passage or opening configured to slideably receive the elongated body, in particular, at least part of the distal end of the elongated body. The recitation “a passage configured to receive the distal end of the elongated body”, as used herein, is meant to encompass that the passage of the coupling element is configured to comprise the distal end of the elongated body. The same meaning can be given to “an opening configured to receive the distal end of the elongated body”.

The recitation “a passage configured to fittingly receive the distal end of the elongated body”, as used herein, is meant to encompass that the passage of the coupling element is configured to fit the distal end of the elongated body. For example, the passage of the coupling element fittingly receives the distal end of the elongated body when the diameter of the passage of the coupling element and the outer diameter of the elongated body are substantially similar. The same meaning can be given to “an opening configured to fittingly receive the distal end of the elongated body”.

The recitation “a passage or opening configured to slideably receive the distal end of the elongated body”, as used herein, is meant to encompass that the distal end of the elongated body, is able to slide into the coupling element.

The passage may have any transverse profile to complement the transverse profile of the distal end of the elongated body. The passage may have any transverse profile such as circular, oval, or polygonal. Preferably, the passage is cylindrical with a circular transverse profile.

The cross-member may be substantially made of any polymeric material which is tolerated by the uterus. Preferably, the cross-member is substantially made of polyethylene or EVA copolymer.

The arms and the coupling element may be made from the same material, for instance from a polymeric material which is tolerated by the uterus. Preferably, the arms and the coupling element are substantially made of polyethylene or EVA copolymer.

The IUD may comprise a radiopaque material. The cross-member may be substantially made of a polymeric material comprising the radiopaque material. The cross-member may comprise one or more markings below or on the surface of the cross-member.

In an embodiment, the radiopaque material may be selected from the group comprising barium, gold, platinum, tantalum, bismuth, silver, titanium, tungsten, palladium, and iodine, or salts thereof. The radiopaque material can be incorporated into the IUD in several ways. Biocompatible non-immunogenic metals such as gold and platinum may be incorporated as a very fine dispersion with particle sizes less than a few micrometers. Other heavy atoms may be incorporated in the form of inorganic salts, such as barium sulfate.

The use of a radiopaque material in the IUD allows localization of the IUD in the intrauterine cavity. This localization is important to control the correct positioning of the IUD after insertion in the uterus and to allow easy removal of the IUD. Such IUDs comprising a radiopaque material can be easily detected using X-ray techniques. X-ray techniques are performed as known by the skilled man in the art.

In a preferred embodiment, the radiopaque material is barium sulfate. For example, the cross-member may comprise barium sulfate in a concentration ranging from about 1% to about 40% by weight, for example, the cross-member may comprise barium sulfate in a concentration ranging from about 5% to about 30% by weight, for example, the cross-member may comprise barium sulfate in a concentration ranging from about 10% to about 25% by weight, for example, the cross-member may comprise barium sulfate in a concentration ranging from about 20% to about 25% by weight. Preferably, the cross-member may comprise barium sulfate in a concentration ranging from about 21% to about 23% by weight.

For instance, the cross-member may be substantially made of alathon, i.e., polyethylene comprising 21-23% by weight of barium sulfate.

The arms and the coupling element may be integrally formed. The arms and the coupling element may be made by injection molding.

The arms of the cross-member may have an outer diameter of about 0.5 mm, 0.6 mm, 0.8 mm, 1 mm, 1.2 mm, 1.4 mm, 1.6 mm, 1.8 mm, 2 mm or a value in the range between any two of the aforementioned values. The arms of the cross-member may have an outer diameter ranging of from about 0.5 mm to about 2 mm, for example, the arms may have an outer diameter ranging of from about 0.8 mm to about 1.5 mm. Preferably, the arms may have an outer diameter of about 1.2 mm.

The coupling element of the cross-member may have a diameter of at least 2 mm, for example, the coupling element may have a diameter of about 3 mm, about 4 mm, about 5 mm, or about 6 mm. Preferably, the coupling element may have a diameter of about 3 mm.

The cross-member may have a length ranging of from about 20 mm to about 36 mm, for example, the cross-member may have a length ranging of from about 24 mm to about 28 mm. Preferably, the cross-member may have a length of about 28 mm.

In an embodiment, the cross-member provides an anchoring point for anchoring of the withdrawal string. The anchoring point provides a point for receiving forces pulling the IUD out of the uterus by the withdrawal string.

In an embodiment, the withdrawal string is anchored to the cross-member. The withdrawal string may be anchored to the cross-member by passing such as threading the withdrawal string through the cross-member, by overmolding the cross-member on the withdrawal string, or by means of an adhesive such as glue or tape. The term anchored may mean secured, attached, connected, or hooked.

The withdrawal string preferably is anchored to the coupling element. The aforementioned anchoring point may be provided on the coupling element. The withdrawal string may be anchored to the coupling element by passing such as threading the withdrawal string through the coupling element, by overmolding the cross-member and/or the coupling element on the withdrawal string, or by attaching the withdrawal string to the coupling element by means of an adhesive such as glue or tape.

For example, the cross-member may be made by injection molding thereby providing a channel in the cross-member, preferably in the coupling element, said channel being configured for passing through the withdrawal string and afterwards, the withdrawal string may be anchored to the cross-member by passing such as threading the withdrawal string through the channel. In a preferred embodiment, the withdrawal string is anchored to the cross-member by an anchoring point in the coupling element that is a bar over which the withdrawal string loops. Preferably, the bar crosses a channel in the coupling element, which channel connects the distal end of the coupling element to the elongated body-receiving passage or opening in the coupling element.

Alternatively, the withdrawal string can be placed in a mold for instance a mold for the manufacture of the coupling element, the mold is closed and the coupling element is produced by overmolding the cross-member on the withdrawal string, following which the cross-member comprising the withdrawal string molded in the coupling element is removed from the mold. A loop of cord may for example be placed in the mold at the position of the coupling element with the two extending portions of the loop of cord extruding from the mold at the passage of the coupling element.

In an embodiment, the cross-member provides a point for pulling the IUD out of the uterus.

In an embodiment, the withdrawal string is anchored to the cross-member and is threaded through the hollow of the stem element and optionally through the hollow of the end piece.

The term “withdrawal string” generally refers to a flexible element that is attached to the proximal end of the stem element or that extrudes from the proximal end of the stem element. Generally, the withdrawal string of an IUD is used to aid the medical practitioner in removing the IUD when it is no longer active or desired and/or to allow the subject to feel if the IUD is still in place. The term “withdrawal string”, as used herein, encompasses an element that holds the cross member and the stem element of the IUD together and that exits from the proximal end of the stem element of the IUD.

The withdrawal string preferably holds the cross-member and the stem element together. For example, the withdrawal string preferably holds the cross-member and the elongated body and optionally the end piece together. The withdrawal string preferably maintains the stem element in an abutting alignment with the cross-member.

In certain embodiments, the withdrawal string may comprise or consist of one strand or fiber, or the withdrawal string may comprise or consist of two or multiple strands or fibers, optionally twisted or braided together. Such withdrawal strings may advantageously provide additional strength such as additional tensile strength.

In an embodiment, the withdrawal string may be a continuous length of cord or thread, or may comprise multiple lengths of cord or thread for instance melted, glued or tied together.

The withdrawal string may be substantially made of a polymeric material, or metallic substance such as an alloy (e.g. nitinol), copper or steel. The withdrawal string may also comprise one or more lengths of threads substantially made of a polymeric material or metallic substance. The length of thread substantially made of a polymeric material preferably extrudes from the stem element, in particular, from the proximal end of the stem element. The length of thread substantially made of metallic substance such as copper preferably assembles the cross-member and the stem element.

The withdrawal string is preferably substantially made of a polymeric material with a higher fusion temperature than the polymeric material of the cross-member.

The withdrawal string is preferably substantially made of a material having a high tensile strength. In an embodiment, the withdrawal string may have a tensile strength of at least 9N, for example, the withdrawal string may have a tensile strength of at least 10N, for example, the withdrawal string may have a tensile strength of at least 11N, for example, the withdrawal string may have a tensile strength of at least 12N.

The withdrawal string may be substantially made of a polymeric material selected from polypropylene, nylon, polyethylene, and polyester.

The withdrawal string may comprise a stop member. The withdrawal string may comprise a stop member configured to maintain the stem element and the cross-member in abutting alignment. The withdrawal string may be provided with a stop member that engages with the proximal end of the stem element such as with the proximal end of the elongated body or with the proximal end of the end piece. The stop member preferably is configured to limit, reduce or abolish movements of the stem element along the withdrawal string. The stop member may be a knot.

The stop member preferably keeps the cross-member and the stem element together. The stop member preferably maintains the cross-member and the stem element in association. The stop member preferably maintains the distal end of the elongated body in association with the passage of the coupling element. The stop member preferably maintains the cross-member and the stem element in association whereby the cross-member and the stem element cannot or can only marginally move along the longitudinal axis of the stem element. The stop member preferably keeps the cross-member and the stem element assembled.

The stop member preferably retains the stem element in position in the cross-member. For example, the stop member may retain the distal end of the elongated body in the passage of the coupling element of the cross-member. The stop member preferably maintains the stem element and the cross-member in a continuous structure.

In order to limit, reduce or abolish movements of the stem element along the withdrawal string, the end piece may be heated to such a temperature that the end piece and the withdrawal string threaded through the end piece are fused together. The end piece fused to the withdrawal string may limit or abolish movements of the stem element along the withdrawal string. The withdrawal string and the end piece may also be glued together.

The withdrawal string may have a diameter ranging of from about 0.1 mm to about 0.4 mm, for example, the withdrawal string may have a diameter ranging of from about 0.2 mm to about 0.3 mm.

The withdrawal string may be single, double, or a loop of cord.

The withdrawal string preferably is a loop of cord, wherein the loop of cord is threaded through a channel in the coupling element and the two extending portions of the loop of cord extrude from the passage of the coupling element such as along the longitudinal axis of the coupling element.

The withdrawal string may also be a loop of cord, wherein the cross-member or coupling element may be overmolded on the loop of cord and the two extending portions of the loop of cord may extrude from the passage of the coupling element such as along the longitudinal axis of the coupling element.

The withdrawal string may also be a cord, wherein the cross-member or coupling element may be overmolded on one end of the cord and the other end of the cord may extrude from the passage of the coupling element such as along the longitudinal axis of the coupling element.

The withdrawal string may also be a cord comprising a knot at one end, wherein the cross-member or coupling element may be overmolded on the knot and the other end of the cord may extrude from the passage of the coupling element such as along the longitudinal axis of the coupling element.

In an embodiment, the IUD has a tensile strength of at least 9N, for example, the IUD has a tensile strength of at least 10N, for example, the IUD has a tensile strength of at least 11N, for example, the IUD has a tensile strength of at least 12N.

In an embodiment, the present IUD may be inserted into the uterine cavity by means of an inserter. Several types of inserters are known in the art. The inserter typically comprises an elongated hollow body configured to at least partially enclose the IUD. Several methods for insertion of the IUD into the intrauterine cavity are known in the art. The type of inserter and the method of insertion may depend on the arms of the present IUD. In case the arms are elongated members, the arms may be folded upwards into the inserter. In case the arms are bent, the arms may remain outside of the inserter and may be folded towards the stem element of the IUD during insertion. Advantageously, due to the design of the IUD, the diameter of the stem element of the IUD is reduced thereby allowing a reduction of the diameter of the inserter. Such an inserter with reduced diameter facilitates the insertion of the IUD in the intrauterine cavity and hence, increases the subject's compliancy.

In an embodiment, the present IUD may be removed from the intrauterine cavity by any method known in the art. Advantageously, the cross-member provides a point for pulling the IUD out of the intrauterine cavity, thereby obviating the need of high tensile strength of the stem element.

A further aspect relates to a cross-member comprising: (a) a coupling element configured for dismountable attachment to a stem element of an IUD, (b) one or more arms attached to the coupling element, and (c) a withdrawal string attached to the coupling element, wherein the coupling element comprises a passage configured to receive an end of the stem element.

Further provided herein is a stem element configured for dismountable attachment to a cross-member of an IUD, the stem element comprises a hollow elongated body, thereby defining a distal end and a proximal end, wherein the elongated body comprises a slow-release polymer and wherein the distal end is configured to fit to a passage of the cross-member.

A further aspect relates to a kit of parts comprising a cross-member and a stem element, wherein the cross member and the stem element are configured to allow for producing an IUD as defined herein.

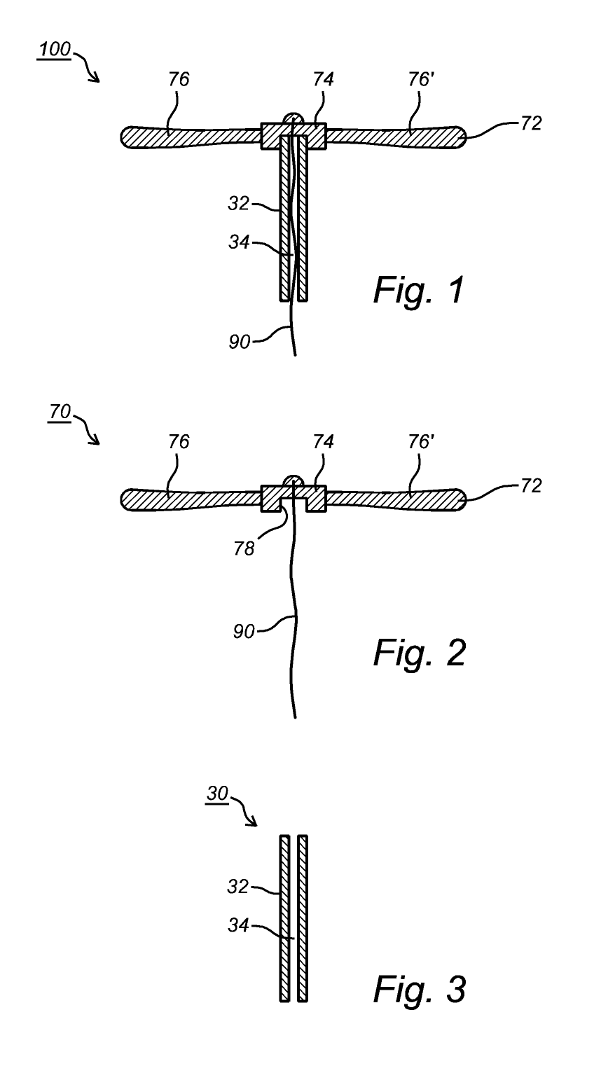

Non-limiting examples of a cross-member, a stem element and an intrauterine device according to the present invention are for instance illustrated in FIGS. 1 to 7.

Referring to FIG. 1, an intrauterine device 100 is shown comprising a stem element, a cross-member, and a withdrawal string 90. The stem element comprises a hollow 34 elongated body 32 comprising a slow-release polymer. The cross-member comprises two arms 76, 76′ attached to a coupling element 74 for dismountable attachment to the elongated body 32. The withdrawal string 90 is anchored to the coupling element 74 and is threaded through the hollow 34 of the elongated body 32. Each arm 76, 76′ comprises an elongated member 72.

Referring to FIG. 2, a cross-member 70 is shown comprising two arms 76, 76′ and a coupling element 74 disposed between the two arms 76, 76′. The withdrawal string 90 is anchored to the coupling element 74. Each arm 76 comprises an elongated member 72. The arms 76, 76′ are attached to the coupling element 74. The two arms 76, 76′ are arranged symmetrically around the longitudinal axis of the coupling element 74. As illustrated in FIG. 2, the arms may have rounded tips. The coupling element 74 is configured for dismountable attachment to an elongated body (not shown) of an IUD. The coupling element 74 comprises a passage or opening 78 configured to receive one end of an elongated body (not shown) of an IUD.

Referring to FIG. 3, a stem element 30 is shown comprising a hollow 34 elongated body 32 comprising a slow-release polymer.

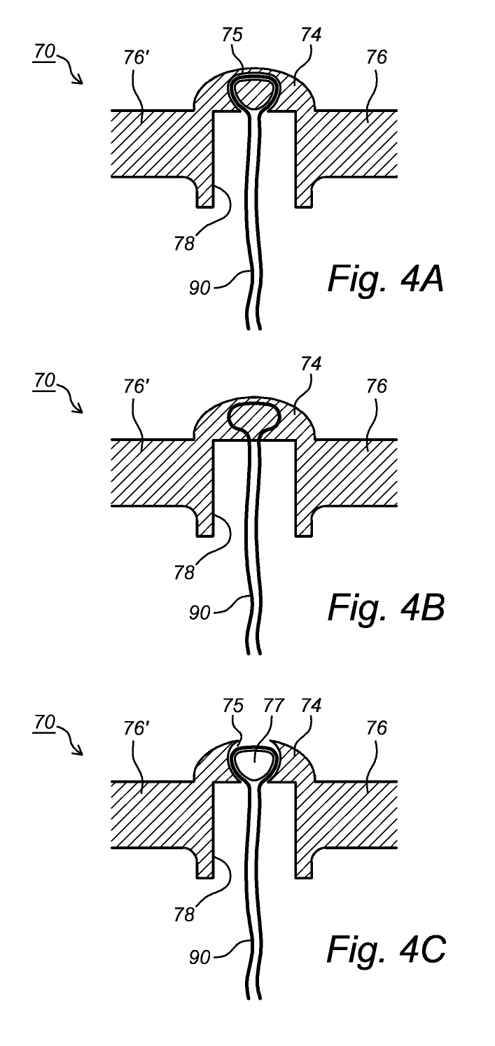

FIG. 4A and FIG. 4B schematically illustrate an enlarged transversal cross-sectional view of a part of a cross-member 70 according to certain embodiments of the invention. The cross-member 70 comprises two arms 76, 76′ and a coupling element 74 disposed between the two arms 76, 76′. The arms 76, 76′ and coupling element 74 are integrally formed, i.e., they are made as one piece. The arms 76, 76′ and coupling element 74 are made from the same material, preferably a polymeric material such as polyethylene. The coupling element 74 is configured for dismountable attachment to the stem element of an IUD. The coupling element 74 comprises a passage or opening 78 configured to receive one end of the elongated body (not shown) of an IUD. The withdrawal string 90 is anchored to the coupling element 74.

As illustrated in FIG. 4A, the withdrawal string 90 may be a loop of cord which is threaded through a channel 75 in the coupling element 74. The withdrawal string is preferably made from a material with a high tensile strength, for instance, the withdrawal string is made of polypropylene. As illustrated in FIG. 4B, the withdrawal string 90 may be a loop of cord which may be anchored to the coupling element 74 by overmolding the coupling element 74 on the loop of cord. The withdrawal string 90 is preferably made form a polymeric material with a higher fusion temperature than the polymeric material of the cross-member, for instance, the withdrawal string 90 is made of polypropylene. In FIG. 4C, the withdrawal string 90 may be a loop of cord which is looped around a bar 77 provided in the coupling element 74. This embodiment is similar to that of FIG. 4A, except the distal end of the coupling element 74 is open in FIG. 4C i.e. the channel 75 connects the distal end of the coupling element 74 with the passage 78.

FIG. 5 schematically illustrates an enlarged transversal cross-sectional view of a part of a stem element 30 according to an embodiment of the invention. The stem element 30 comprises an elongated body 32 comprising a slow-release polymer such as EVA, PU, or silicone. The elongated body 32 comprises a hollow or lumen 34 that spans the length of the elongated body 32. The distal end 31 of the elongated body 32 is configured to fit in the passage 78 of the cross-member 70.

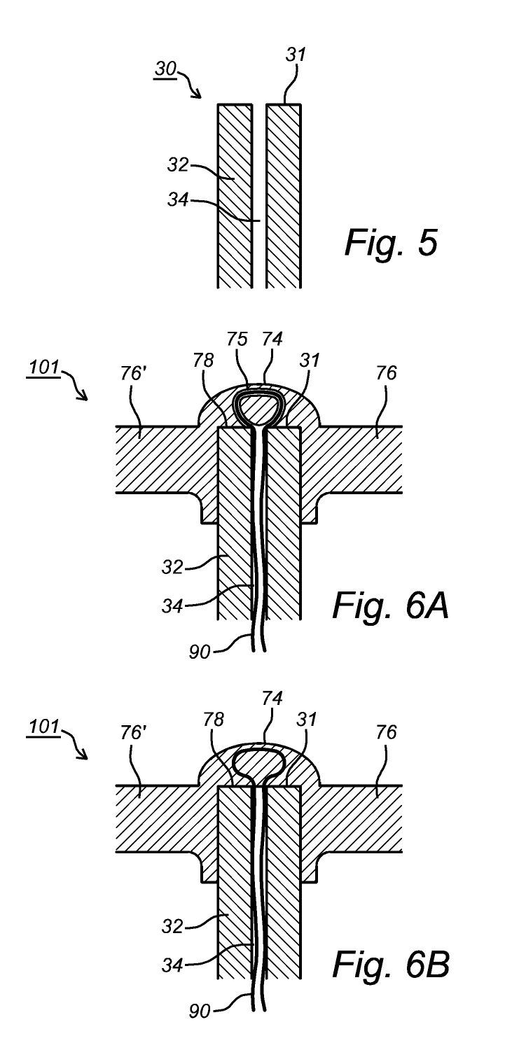

FIG. 6A and FIG. 6B schematically illustrate an enlarged transversal cross-section of a part of an IUD 101 according to certain embodiments of the invention. The IUD 101 comprises a stem element comprising a hollow 34 elongated body 32 comprising a slow-release polymer. The IUD 101 further comprises a cross-member comprising two arms 76, 76′ attached to a coupling element 74 for dismountable attachment to the stem element. The coupling element comprises a passage 78 configured to receive the distal end 31 of the elongated body 32. As illustrated, the passage or opening 78 fittingly receives the distal end 31 of the elongated body 32. The IUD further comprises a withdrawal string 90 anchored to the coupling element 74 and threaded through the hollow 34 of the elongated body 32.

As illustrated in FIG. 6A, the withdrawal string 90 may be a loop of cord which is threaded through a channel 75 in the coupling element 74. As illustrated in FIG. 6B, the withdrawal string 90 may be a loop of cord which is anchored to the coupling element 74 by overmolding the coupling element 74 on the loop of cord.

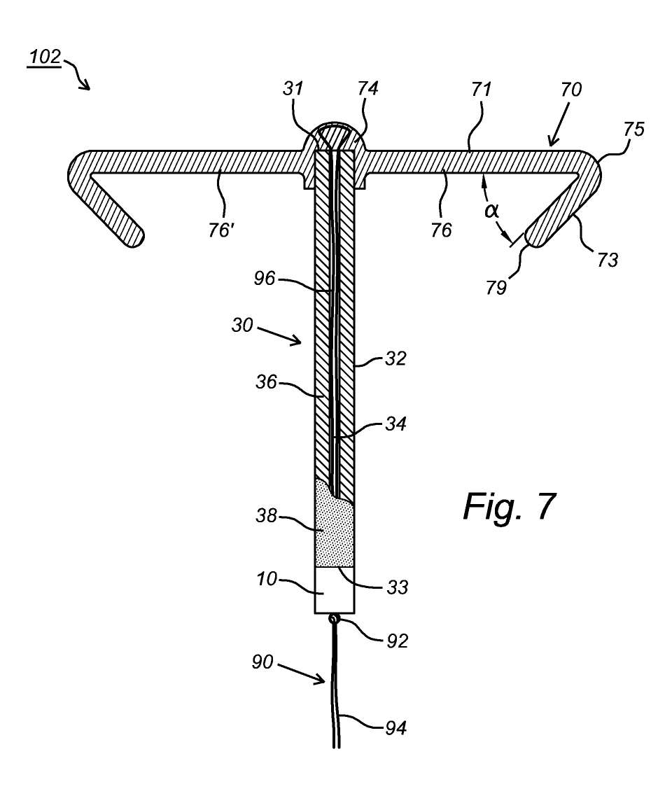

FIG. 7 schematically illustrates a transversal cross-section of an IUD 102 according to an embodiment of the invention.

The intrauterine device 102 comprises a stem element 30 comprising an elongated body 32 comprising a slow-release polymer. The elongated body 32 comprises a hollow 34 provided as a lumen along the longitudinal axis of the stem element 30. The elongated body 32 defines a distal end 31 and a proximal end 33. The elongated body 32 comprises a polymeric core 36 comprising a slow-release polymer and the active pharmaceutical ingredient and an outer membrane 38 for controlling the diffusion of the active pharmaceutical ingredient from the polymeric core 36. The stem element 30 further comprises an end piece 10 preferably substantially made from polyethylene.

The IUD 102 also comprises a cross-member 70 comprising a coupling element 74 and two arms 76, 76′ attached to the coupling element 74 and symmetrically arranged around the longitudinal axis of the coupling element 74. The extremity of each of the two arms 76, 76′ is bent through an angle greater than 90° so that the extremities point to the stem element 30. The extremity of each of the two arms 76, 76′ preferably is bent through an angle of 145°. Each arm thus comprises three parts, i.e. a first part 71, a second part or extension 73, and a bent piece 75 therebetween. The angle α between the first part 71 and the second part or extension 73 may be less than 90° and preferably 35°. Each arm 76 and 76′ has a rounded tip 79.

The intrauterine device 102 further comprises a withdrawal string 90 which is attached to the coupling element 74 and is threaded through the hollow 34 of the elongated body 32. The withdrawal string 90 is further threaded through the end piece 10. The withdrawal string is a loop of cord comprising a part 96 anchored to the cross-member and threaded through the hollow 34 of the elongated body and through the end piece 10 and a part 94 extending from the hollow of the stem element 30. The withdrawal string 90 is substantially made of polypropylene. The withdrawal string puts the cross-member, the elongated body and the end piece together. The withdrawal string comprises a knot 92 engaging with the proximal end of the stem element, in particular with the proximal end of the end piece 10. The knot 92 maintains the cross-member, elongated body, and the end piece in association. As illustrated in FIG. 7, the cross-member, elongated body, and end piece form a continuous structure.

1. An intrauterine device, IUD, comprising a stem element, a cross-member, and a withdrawal string, wherein:
the stem element comprises a hollow elongated body;
the cross-member comprises a coupling element configured for dismountable attachment to the stem element, and one or more arms attached to the coupling element; and
the withdrawal string is anchored to the cross-member and is threaded through the hollow of the stem element, characterized in that the coupling element comprises a passage configured to fittingly receive a distal end of the hollow elongated body of the stem element.

2. The IUD according to claim 1, wherein the withdrawal string is anchored to the coupling element.

3. The IUD according to claim 1, wherein the coupling element comprises a passage configured to slideably receive a distal end of the hollow elongated body of the stem element.

4. The IUD according to claim 1, wherein the stem element is essentially rigid.

5. The IUD according to claim 1, wherein the withdrawal string comprises a stop member configured to maintain the stem element coupled to the coupling element of the cross-member.

6. The IUD according to claim 5, wherein the stop member is a knot.

7. The IUD according to claim 1, wherein the IUD is one that elutes a composition.

8. The IUD according to claim 7, wherein the stem element is at least partially formed from a polymer comprising the composition.

9. The IUD according to claim 8, wherein the polymer is adapted to prolong and/or control the release of the composition from the IUD.

10. The IUD according to claim 8, wherein the polymer is selected from the group consisting of ethylene/vinyl acetate (EVA) copolymer, polyurethane (PU), and silicone.

11. The IUD according to claim 8, wherein the polymer is formed as a polymeric core comprising the composition, disposed with an outer membrane configured to control release of the composition.

12. The IUD according to claim 7, wherein the composition comprises at least one active pharmaceutical ingredient.

13. The IUD according to claim 1, wherein the elongated body is cylindrical and has an outer diameter equal to or less than 2.2 mm.

14. The IUD according to claim 1, wherein the IUD has a tensile strength of at least 9N.

15. The IUD according to claim 2, wherein the withdrawal string comprises a stop member configured to maintain the stem element coupled to the coupling element of the cross-member.

16. The IUD according to claim 15, wherein the stop member is a knot.

17. The IUD according to claim 9, wherein the polymer is selected from the group consisting of ethylene/vinyl acetate (EVA) copolymer, polyurethane (PU), and silicone.

18. The IUD according to claim 17, wherein the polymer is formed as a polymeric core comprising the composition, disposed with an outer membrane configured to control release of the composition.

19. The IUD according to claim 9, wherein the polymer is formed as a polymeric core comprising the composition, disposed with an outer membrane configured to control release of the composition.

20. The IUD according to claim 10, wherein the polymer is formed as a polymeric core comprising the composition, disposed with an outer membrane configured to control release of the composition.
